(19) United States
(12) Patent Application Publication
(10) Pub. No.: US 2005/0209204 A1
(43) Pub. Date: Sep. 22, 2005

(54) Treatment of age-related lung abnormalities using estrogen and/or retinoids

(75) Inventors: Donald Massaro, Washington, DC (US); Gloria D Massaro, Washington, DC (US)

Correspondence Address: PILLSBURY WINTHROP SHAW PITTMAN, LLP, P.O. BOX 10500, MCLEAN, VA, 22102, US

(73) Assignee: GEORGETOWN UNIVERSITY, WASHINGTON D.C., DC (US)

(21) Appl. No.: 10/513,136

(22) PCT Filed: May 2, 2003
(86) PCT No.: PCT/US03/13717

§ 371 Date: May 1, 2005

Related U.S. Application Data
(60) Provisional application No. 60/376,841, filed on May 2, 2002.

(51) Int. Cl. A61K 31/56; A61K 31/203

(52) U.S. Cl. 514/171; 514/559

(57) ABSTRACT

Methods of treating diseases or age disorders involving loss of alveoli, lung recoil or elasticity and gas exchange using RARα agonists and estrogen therapies are provided.

### CROSS-REFERENCES TO RELATED APPLICATIONS

This application claims priority to U.S. Provisional Application No. 60/376,841 filed May 2, 2002, which is incorporated herein in its entirety.

### FIELD OF THE INVENTION

The invention relates to the improvement of lung function in aged but otherwise normal individuals by the use of estrogen replacement therapy and/or the administration of retinoic acid or another retinoid which specifically binds retinoid acid receptors (RARS α, β, or γ). These therapies are useful in enhancing alveolar development and function as well as lung elasticity or lung recoil. These combination therapies are also useful for treatment of other lung disorders, e.g., COPD, coronary artery disease and asthma. Also, the invention relates to the use of RARα agonists or compounds which pharmacologically upregulate RARα protein in the lung of individuals with diseases that cause alveolar destruction such as identified above or in aged individuals who experience loss of gas-exchange function. The invention also relates to the screening of individuals with loss of gas-exchange function to assess whether they have a genetic or acquired defect that affects RARα expression.

### BACKGROUND OF THE INVENTION

All-trans retinoic acid (ATRA) is a multifunctional modulator of cellular behavior, having the potential to alter both extracellular matrix metabolism and normal epithelial differentiation. In the lungs, ATRA to modulates various aspects of lung differentiation by interacting with specific retinoic acid receptors (RAR) and RXRS that are selectively expressed temporally and spatially. Coordinated activation of RAR alpha, RAR.beta and RAR.gamma. has been associated with lung branching, gene activation of tropoelastin in neonatal rats alveolar formation septation in fetal, neonatal, and adult rats and mice.

During alveolar septation, retinonic acid storage granules (retinyl-esters) increase in the fibroblastic mesenchyme surrounding alveolar walls (Liu et al; Am. J. Physiol. 265: L430-L437, 1993; McGowan et al Am. J. Physiol. 269: L463-L472, 1995), and cellular retinonic acid binding protein I expression in the lung peaks (Ong, D. E. and Chytil, F., Proc. Natl. Acad. of Sciences 73: 3976-3978, 1976; Grummer, M. A., Thet, L. and Zachman, R. D., Pediatr. Pulm. 17: 234-238, 1994). Depletion of these retinyl-ester stores parallels the deposition of new elastin matrix and sepation. In support of this concept, Massaro and Massaro (Massaro, D. and Massaro, G., Am. J. Physiol. 270, L305-L310, 1996) demonstrated that postnatal administration of retinoic acid increases the number of alveoli in rats. Treatment of newborn rat pups with dexamethasone inhibits the process of septation in the lungs. This effect can be prevented by supplemental treatment with retinoic acid. Furthermore, the capacity of dexamethasone to prevent the expression of CRBP and subsequent alveolar septation in developing rat lung was abrogated by ATRA.

Recent studies demonstrated that ATRA can induce formation of new alveoli and return elastic recoil to near normal in animal models of emphysema (Massaro, D. and Massaro, G., Nature Med. 3: 675-677, 1997; “Strategies to Augment Alveolization,” National Heart, Lung, and Blood Institute, RFA: HL-98-011, 1998.). However the mechanism by which this occurs remains unclear.

Retinoids are a class of compounds structurally related to vitamin A, comprising natural and synthetic compounds. Several series of retinoids have been found clinically useful in the treatment of dermatological and oncological diseases. All-trans retinoic acid (ATRA) and its other naturally occurring retinoid analogs (9-cis retinioc acid, all-trans 3-4 didehydro retinioc acid, 4-oxo retinoic acid and retinol) are pleiotrophic regulatory compounds that modulate the structure and function of a wide variety of inflammatory, immune and structural cells. They are important regulators of epithelial cell proliferation, differentiation and morphogenesis in lung. Retinoids exert their biological effects through a series of nuclear receptors that are ligand inducible transcription factors belonging to the steroid/thyroid receptor superfamily.

The retinoid receptors are classified into two families, the retinoic acid receptors (RARs) and the retinoid X receptors (RXRs), each consisting of three distinct subtypes (.alpha., .beta., and .gamma.). Each subtype of the RAR gene family encodes a variable number of isoforms arising from differential splicing of two primary RNA transcripts. ATRA is the physiological hormone for the retinoic acid receptors and binds with approximately equal affinity to all the three RAR subtypes. ATRA does not bind to the RXR receptors; instead, for these receptors, 9-cis retinoic acid is the natural ligand.

In many non-pulmonary tissues, retinoids have anti-inflammatory effects, alter the progression of epithelial cell differentiation, and inhibit stromal cell matrix production. These properties have led to the development of topical retinoid therapeutics for dermatological disorders such as psoriasis, acne, and hypertrophic cutaneous scars. Other applications include the control of acute promyelocytic leukemia, adeno- and squamous cell carcinoma, and hepatic fibrosis. However, therapeutic use of retinoids outside of cancer is limited due to the relative toxicities observed with the naturally occurring retinoids, ATRA and 9-cis RA. These natural ligands are non-selective and, therefore, have pleiotrophic effects throughout the body, which are often toxic. Recently various retinoids have been described that interact selectively or specifically with the RAR or RXR receptors or with specific subtypes (.alpha., .beta., .gamma.) within a class. Using these novel retinoids, the transrepression of AP-1 and transactivation activities of retinoids have been separated. (Li, J. J. et al, Cancer Research, 56: 483489 (1996); Fanjul, A. et al., Nature, 372: 107-111 (1994); Schule R. et al., PNAS, 88: 6092-6096 (1991); Nagpal et al., Journal of Biological Chemistry 270: 923-927 (1995)). In addition, the receptor selective compounds have shown reduced general toxicity in vitro and in vivo. (Chandraratna, R., J. Am. Acad. Dermatology, 39: S149-S152, 1998; Look, J. et al., Am. J. Physiol. 269: E91-E98, 1995).

### SUMMARY OF THE INVENTION

In one aspect the invention provides methods of treating an age-related lung defect or condition by the administration of estrogen an/or a retinoid that specifically interacts with at least one retinoid acid receptor (RAR).

In another aspect the invention provides methods of treating pulmonary diseases such as COPD, asthma and persons with age-related loss of alveoli or gas-exchange function by administration of an RAR-α agonist. In another aspect the invention provides a method of identifying subjects eligible for such treatment by screening for a genetic or acquired defect involving RARα function.

In a preferred aspect the invention provides a method of treating age-related lung conditions or defects, especially to improve lung elasticity or recoil and/or alveolar development and/or regeneration by a synergistic combination therapy comprising the administration of an estrogen and a retinoid that specifically interacts with at least one RAR selected from RARα, RARβ and RARγ, wherein said estrogen and retinoid are administered separately or in combination and in either order

### DETAILED DESCRIPTION OF THE INVENTION

Definitions

As used herein the following phrases or words have the meaning below. In all other instances words or phrases are to be given that ordinary meaning as they would be construed by one having ordinary skill in the art.

Aged individual—In this invention this refers to an individual 45 years or older, more preferably 50 or 55 years or older, and still more preferably 65 years or older.

Normal aged individual—In this invention this refers to an individual with normal age-related lung defects, e.g., loss of lung recoil or elasticity or loss of alveolar function, number, regeneration or development. These Individuals do not possess a non-aged related lung condition such as emphysema, COPD or cystic fibrosis.

Combination Therapy—This refers to a therapy wherein at least two compounds are administered to treat a condition, wherein said compounds are administered separately or in combination, and in either order.

Synergistic Combination—This refers to a treatment involving the administration of at least two compounds that provides for enhanced therapeutic effect, e.g. enhanced alveolar function, number, regeneration, gas exchange, lung recoil or elasticity than either compound taken individually.

Retinoid-refers to a class compounds structurally related to vitamin A comprising natural and synthetic compounds. These compounds such as ATRA and structurally related analogs (9-C15) retinoid acid, all-trans 3-4 didenydium retinoid acid, 4-oxa retinoid acid and retinol are pleiotropic regulatory compounds that regulate the structure and function of a wide variety of inflammatory, immune and structural cells. They are important regulators of epithelial cell proliferation, differentiate and lung morphogenous.

Retinoid receptors are in two families, the retinoid acid receptor (RAR γ) and the retinoid X receptor(RXR γ), and each comprises three subfamilies RARα, β, and γ and RαR, β, and γ.

RARα agonist is a compound that agonizes the activity of the RARα receptor. Examples of such compounds are known in the art and include by way of example the agonist compounds disclosed in U.S. Pat. Nos. 6,355,669; 6,187,950; 6,075,032; 6,071,924; 6,063,797; and 5,759,785, all incorporated by reference in their entirety hrein.

Additionally, compounds which agonize the RAR alpha receptor can be identified as the RAR alpha receptor has been cloned and is reported in U.S. Pat. No. 5,571,692 also incorporated by reference in its entirety herein. “Methods for screening defective abnormal RARα activity or expression, e.g., reduced or increased levels of expression of RARα or an RARα having an aberrant sequence. Mutations that affect RARα function are known and are disclosed e.g., in U.S. Pat. Nos. 6,093,873, 6,030,794 and 6,031,149 also incorporated by reference herein.

In one embodiment age-related loss of lung elasticity and/or recoil or alveolar development according to the invention will be treated or prevented by the administration of an estrogen compound or estrogen antagonist in an amount effective to treat age-related lung dysfunction.

Examples of suitable estrogen and estrogen analogs and estrogen agonists are well known in the art and include estradiol, phytoestrogens, ethnyl estradiol, mestranol, 17 beta-estradiol, 17 alpha-estradiol, tamoxifen derivatives such as 4-hydroxytamoxifer,

The estrogen compound or estrogen antagonist will be administered by any route that provides for effective treatment including topical, subcutaneous, inhalation, intramuscular, intravenous, intranasal, oral, rectal, vaginal, etc. The compound may be administered alone or in combination with other estrogen compounds or antagonists, or other hormones such as progestin. The dosage will depend on the particular estrogen compound, and the particular patient.

Typically, the dosage of estrogen compound will range from about 0.001 to 200 mg/day, more typically from about 0.01 to 20 mg/day and most typically from about 0.1 to 10 mg/day.

As estrogen therapy can sometimes cause adverse side-effects the subject will be monitored to ensure that this does not occur. For example, any increased urine excretion, weight gain, changes in breast or uterine tissues will be monitored. If this is observed, the particular design of the estrogen compound may be reduced or the particular compound changed.

Suitable estrogens and analogs and estrogen antagonists may be isolated from natural sources, or synthesized by chemical or recombinant methods. Many recombinant and synthetic estrogens and estrogen agonists are commercially available and are the subject of numerous issued patents.

See e.g., U.S. Pat. Nos. 6,358,943, 6,355,670, 6,355,623, 6,355,630,6,352,970, 6,331,562, 6,326,366, 6,323,190, 6,316,494, 6,274,618 all of which are incorporated by reference in their entirety and are exemplary of issued patents relating to estrogen compounds and agonists suitable for human therapy.

As discussed, the subject who is treated is male or female and will exhibit age-related lung abnormalities. This may be determined e.g., by a test of lung function after prolonged exercise in an otherwise normal subject, i.e., who does not have a lung disorder such as emphysema or COPD.

In a preferred embodiment, the subject invention will comprise the treatment of male or female subjects having age-related lung dysfunction, e.g., related exercise tolerance, reduced lung elasticity or recoil, or reduced alveolar regeneration, repair or development by a combination therapeutic regimen including the administration of an estrogen, estrogen analog or estrogen agonist and a compound that binds a retinoic acid receptor and promotes pulmonary function. Examples thereof include RARα, RARβ, and RARγ agonists and antagonists such as retinoic acid, all trans retinoic acid, 13-Cis retinoic acid, 4HPR, 9-Cis retinoic acid, other esters and analogs of retinoic acid, retinoic acid mimetic anilldes (see e.g., U.S. Pat. No. 6,319,939; et al). Preferably, an RAR agonist will be administered that up-regulates RARα protein in the lung of individuals with loss of alveolar destruction due to diseases such as COPD, asthma or emphysema or age that results in loss of gas-exchange function or loss of lung recoil.

In another preferred embodiment the retinoid will comprise an RARγ selective agonist such as are described in U.S. Pat. No. 6,300,350, issued to Belloni et al. Many other retinoids which may be useful in the subject combination therapy are known in the art. Examples thereof are disclosed in U.S. Pat. Nos. 6,339,107, 6,319,939, 6,291,508, 6,258,811, 6,083,977, 6,030,964, 5,968,981, 5,962,534, and others, all which are incorporated by reference in their entirety.

As with the estrogen, the retinoid compound can be administered by different modes of administration, e.g., oral, intravenous, intramuscular, subcutaneous, inhalation, intranasal, vaginal, rectally, transdermal, etc. In a preferred embodiment the retinoid will be administered by use of a liposomal aerosol delivery system to the lungs as described in U.S. Pat. No. 6,334,999 issued to Gilbert et al.

The dosage of the retinoid compound, e.g., ATRA will typically range from about 0.01 to 100 mg/day, more preferably about 0.1 to 10 mg/day, in order to achieve a concentration in lung tissue ranging from about 0.05 to 20 mg/day lung tissue.

The retinoid and the estrogen may be administered separately or in combination, in either order, in amounts effective to prevent or treat age-related lung dysfunction. It is anticipated that this will yield a synergistic effect on lung function.

In order to illustrate the invention, the following examples are provided.

### EXAMPLES

Treatment of Age Male Subject with Estrogen/Retinoid Treatment

A 65 year old male subject with age-related lung of lung elasticity and alveolar function and development is administered 5 mg/day of all-trans retinoic acid and 10 mg/day of estradiol. The two are administered separately, with the retinoid acid administered in a liposomal aerosol delivery system as described in U.S. Pat. No. 6,334,999, and the estradiol administered orally. A month after treatment and at the start of treatment lung function is evaluated. Efficacy of treatment is determined based on enhanced lung elasticity or recoil and increased alveolar function and development.

Treatment of COPD with RARα Agonist and Estrogen

A patient with COPD is treated with an RARα agonist after genetic testing reveals that RARα expression is impaired relative to normal individuals. Particularly, an agonist compound disclosed in U.S. Pat. No. 6,355,669 is administered at a dosage of 5 mg/day in combination with estrogen administered at a dosage of 10 mg/day.

1. A method of improving lung function in an aged but otherwise normal individual by administering, an effective amount of an estrogen and/or an effective amount of a retinoid that specifically binds to a retinoid acid receptor (RAR).

2. The method of claim 1 wherein an aged individual is an individual 50 years or older.

3. The method of claim 1 wherein are aged individual is an individual 55 years or older.

4. The method of claim 1 wherein said individual does not have any lung disease or non-age associated lung condition.

5. The method of claim 1 wherein said individual is male.

6. The method of claim 1 wherein said individual is female.

7. The method of claim 5 wherein said treatment includes the administration of estrogen or an analog thereof.

8. The method of claim 6 wherein said treatment includes the administration of estrogen or an analog thereof.

9. The method of claim 1 which is to improve exercise to tolerance in an aged individual.

10. The method of claim 1 which is to promote alveolar regeneration and/or development in an aged individual.

11. The method of claim 1 which comprises the administration of an estrogen and a retinoid that specifically binds to an RAR.

12. The method of claim 11 wherein said RAR is RARα, RARβ or RARγ.

13. The method of claim 11 wherein said retinoid is retinoic acid.

14. The method of claim 13 wherein said retinoid acid is all trans retinoic acid (ATRA).

15. The method of claim 11 wherein said retinoid is an RARα agonist.

16. The method of claim 11 wherein said retinoid is an RARβ agonist.

17. The method of claim 11 wherein said retinoid is an RARγ agonist.

18. The method of claim 11 wherein said estrogen and retinoid are administered separately.

19. The method of claim 11 wherein said estrogen and retinoid are administered in combination.

20. The method of claim 18 wherein said estrogen is administered prior to the retinoid.

21. The method of claim 18 wherein said estrogen is administered after the retinoid.

22. The method of claim 1 wherein the estrogen and/or retinoid are administered orally.

23. The method of claim 1 wherein the retinoid is administered via inhalation or intranasally.

24. The method of claim 1 wherein the estrogen and/or retinoid are administered via injection.

25. The method of claim 1 which includes the administration of more than a retinoid that specifically interact with different RARs.

26. The method of claim 1 wherein includes the administration of an RAR gamma selective agonist.

27. A method of treating a lung disorder involving alveolar destruction or loss of gas-exchange function by administering an effective amount of an RAR alpha agonist or a compound which up-regulates RARα protein.

28. The method of claim 27 wherein the disease is COPD.

29. The method of claim 27 wherein the disease is asthma, coronary artificial disease, or asthma.

30. The method of claim 27 wherein said RARα agonist is administered in combination with an estrogen.

31. The method of claim 27 wherein said subject has age-related loss of alveoli or gas-exchange function.
